(19) United States
(12) Patent Application Publication
(10) Pub. No.: US 2022/0009866 A1
(43) Pub. Date: Jan. 13, 2022

(54) SYSTEMS AND METHODS FOR EXTRACTING AND ISOLATING BIO-OILS AND POWDERS FROM BIOMASS USING HYDRAULIC HAMMERING

(71) Applicant: Antares Agriculture Group, LLC, Golden, CO (US)

(72) Inventors: Roger E. Knight, Golden, CO (US); Clint Cornelius, Milliken, CO (US); Brent Cornelius, Milliken, CO (US)

(73) Assignee: Antares Agriculture Group, LLC, Golden, CO (US)

(21) Appl. No.: 17/354,287

(22) Filed: Jun. 22, 2021

Related U.S. Application Data
(60) Provisional application No. 63/042,766, filed on Jun. 23, 2020.

(51) Int. Cl. C07C 37/70; C07C 39/23; B01D 11/02; B01D 25/12

(52) U.S. Cl. CPC C07C 37/70; C07C 39/23; B01D 25/12; B01D 11/0292; B01D 11/0265

(57) ABSTRACT

Methods for obtaining a cannabinoid powder comprising a filtered CBD composition including (a) providing a biomass feedstock comprising a portion of Cannabis sativa plant; (b) extracting by sonification of the biomass feedstock at least a sonification terpene composition; and (c) filtering the sonfiication terpene composition using a pressure filter to produce a filtered CBD composition and a filtered terpene mixture.

### CROSS-REFERENCE TO RELATED APPLICATIONS

The present application claims the benefit under 35 U.S.C. § 119(e) of U.S. Provisional Patent Application Ser. No. 63/042,766, entitled “SYSTEMS AND METHODS FOR EXTRACTING AND ISOLATING BIO-OILS AND POWDERS FROM BIOMASS USING HYDRAULIC HAMMERING,” filed on Jun. 23, 2020, the content of which is incorporated herein by reference in its entirety.

### FIELD OF THE INVENTION

The present invention relates to an ultrasonic method for extraction and isolation of bio-oils, bio powders, and related ultrasonic apparatus.

### BACKGROUND OF THE INVENTION

Raw input or biomass can be used to prepare useful materials, including bio-oils, bio-powder or bio-salve. Generally accepted processes for extracting bio-oils from biomass typically include the use of harsh chemicals in multiple steps. For example, harsh chemicals are often used as solvents or surfactants during the extraction process.

Various attempts have been made to improve oil extraction. For instance, conventional processes typically involve a labor-intensive pre-treatment to isolate the buds (all seeds and plant material removed) by hand or fractionation step of the raw biomass to prepare the feedstock in advance of the extraction steps. The result is a product mixture of various chemicals and compounds, including pre-treatment compounds.

As such, there exists a need for improved methods for extracting materials such as bio-oils from raw biomass materials.

### SUMMARY OF THE INVENTION

The present disclosure provides improved methods for extracting bio-oil powders from raw biomass feedstock, which methods yield a compositionally stable bio-oil powder product profile. In certain embodiments, the methods of the disclosure may be used to extract bio-oil-powders from raw biomass feedstock without chemical pretreatment of the feedstock biomass. In further embodiments, the methods may be used to extract cannabinoid-rich extract oils or powders from cannabis plant feedstock materials, including hemp feedstock materials.

In certain embodiments, the methods use hydraulic hammering to extract bio-oils from feedstock biomass. By way of example, in accordance with embodiment of the disclosure, the hydraulic hammering may utilize ultrasonic energy such as sound waves to extract the bio-oil.

Accordingly to an aspect of the disclose, a method is provided for obtaining bio-oil powders, e.g., a cannabinoid powder comprising a filtered CBD composition, the method including (a) providing a biomass feedstock comprising a portion of Cannabis sativa plant; (b) extracting by sonification of the biomass feedstock at least an sonification terpene composition; and (c) filtering the sonification terpene composition using a pressure filter to produce a filtered CBD composition and a filtered terpene mixture, and (d) forming the cannabinoid powder from the filtered CBD composition.

### BRIEF DESCRIPTION OF THE DRAWINGS

The invention is further illustrated by the following drawings, none of which are intended to be construed as necessarily limiting the invention.

FIG. 1 is an exemplary a flow diagram of a method for extracting bio-oil powder from a biomass feedstock according to an aspect of the disclosure.

FIG. 2 is a first exemplary schematic of a batch process system and related method for extracting bio-oil powder from a biomass feedstock in accordance with an aspect of the disclosure.

FIG. 3 is a second exemplary schematic of a continuous flow system and related method for extracting bio-oil powder from a biomass feedstock according to an aspect of the disclosure.

FIGS. 4-6 are images of exemplary results for analyses of cannabinoid profiles in accordance with an aspect of the disclosure.

FIG. 7 is an image of exemplary results for an analysis of the terpene profile according to an aspect of the disclosure.

It should be understood that the various aspects are not limited to the arrangements, instrumentality, units, and/or streams shown in the drawings.

### DETAILED DESCRIPTION OF THE INVENTION

As discussed in further detail herein, the present disclosure provides systems and methods for extracting bio-oil powders from raw biomass feedstocks using sonification. In certain aspects, it has been surprising found that the methods of the disclosure may be used to extract bio-oil powders from raw biomass feedstock without the need to chemically pretreatment the feedstock biomass. For example, in accordance at least one aspect of the disclosure, bio-oil powders may be extracted from a biomass feedstock comprised of whole plant material that has been only mechanically processed for size reduction.

In certain embodiments, the methods use hydraulic hammering to extract bio-oils from feedstock biomass. By way of example, in accordance with embodiment of the disclosure, the hydraulic hammering may utilize ultrasonic energy such as sound waves to extract the bio-oil. In certain embodiments, the ultrasonic energy may comprise sonification.

In accordance with the disclosure, the biomass feedstock may be derived from any suitable plant materials that contain oils, e.g., essential oils, desired for extraction. In certain embodiments, the biomass feedstock is cannabis plant materials, such as hemp. However, the disclosure is not so limited and any suitable plant feedstock material may be used in connection with the methods and apparatus disclosed herein.

Generally, as used herein, cannabinoid(s) refers to any of the various chemical constituents of hemp or cannabis. Cannabidiol (CBD) is a naturally occurring compound found in the Cannabis sativa plant, derived from the hemp plant. CBD is one of more than a hundred “phytocannabinoids,” which are unique to cannabis and are traditionally believed to provide the cannabis plant with its therapeutic profile.

In accordance with an aspect of the disclosure, a method is provided for extracting bio-oil from a biomass feedstock. The method may include the operation and/or use of systems further described below.

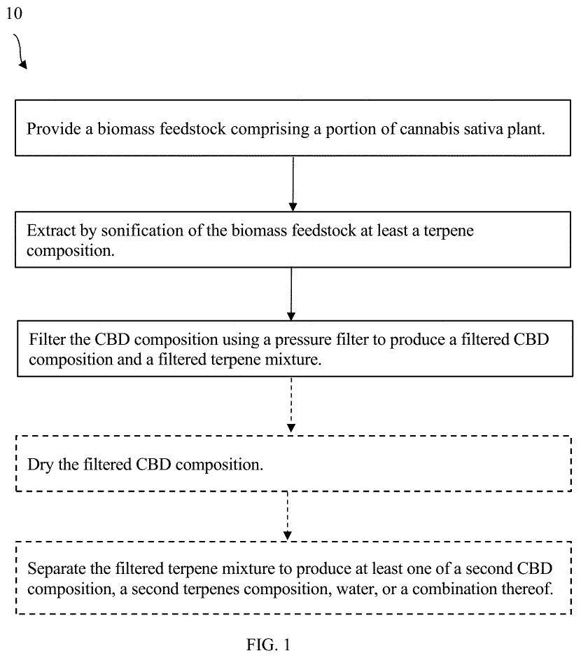

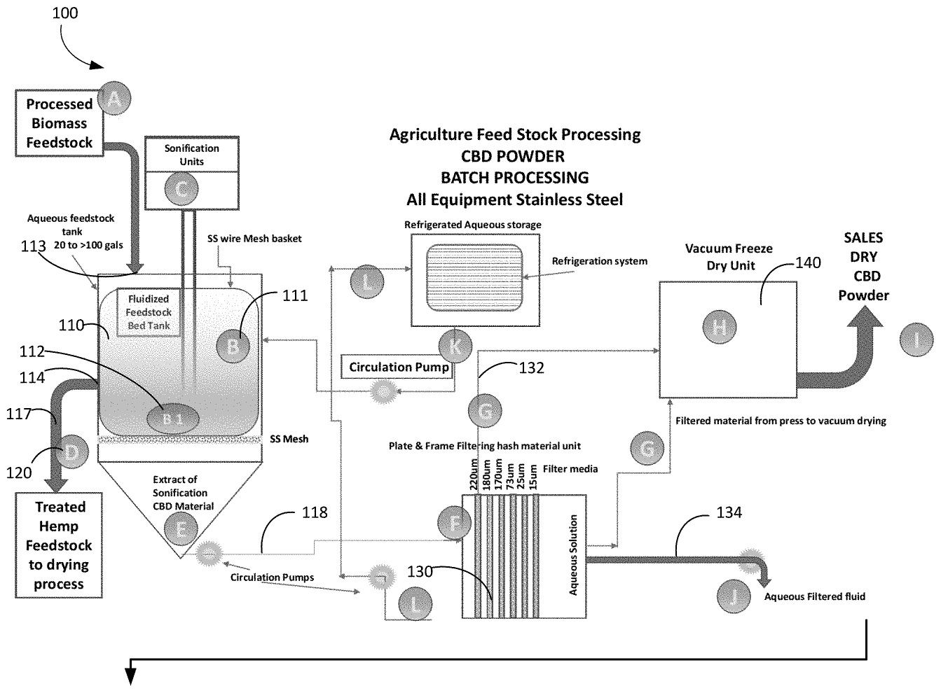

More specifically, with reference to FIG. 1, method 10 generally includes: (a) providing a biomass feedstock comprising a portion of Cannabis sativa plant; (b) extracting by sonification from the biomass feedstock at least a sonification terpene composition; (c) filtering the sonification terpene composition using a pressure filter to produce a filtered CBD composition and a filtered terpene mixture; and (d) forming the cannabinoid powder from the filtered CBD composition. In some cases, the method further includes: (e) drying the filtered CBD composition of (c) and/or (f) separating the filtered terpene mixture of (c) to produce at least one of a second CBD composition, a second terpenes composition, water, or a combination thereof.

As described in further detail herein, the biomass feedstock may be prepared prior to loading into the sonification reactor chamber by any suitable mechanical size reduction methodology. In certain embodiments, the biomass feedstock may be cut into small pieces using a standard grinder and hopper to move the biomass feedstock into the sonification reactor chamber. Again, in certain aspects, it has been surprising found that the methods of the disclosure may be used to extract bio-oils from raw biomass feedstock without the need to chemical pretreatment the feedstock biomass. More specifically, in accordance with the methods of the disclosure, bio-oils may be extracted from raw biomass feedstock comprised of whole plant material, only mechanically processed for size reduction.

As described in further detail herein, the sonification reactor chamber may operate under suitable temperatures, frequencies and volume ratios to result in biomass conversion of the biomass feedstock into sonification oil and terpenes. By way of non-limiting example, temperatures may range from about 20° F. to about 100° F., dependent on biomass feedstock analysis, frequencies range from 20 KHz to 30 KHz, power rating percentage and processing ratios between 25% to 75% biomass/water. Operating frequencies may generally be from the composition of the biomass feedstock analysis.

In certain embodiments, the prepared biomass feedstock may be fed into a suitable loading mechanism, e.g., a rotating valve, which then feeds the feedstock into the sonification chamber. The biomass water mixture is agitated using mechanical of fluid to ensure suspension and biomass contact time with the ultrasonication probe.

In certain embodiments, as the biomass feedstock passes through the sonification reactor chamber, the feedstock is sonically treated to form a cannabinoid volatile product and a biomass product. The sonification volatile product includes solid phase and a liquid phase. Gravity and centrifugal force within the sonification reactor chamber facilitate movement of the sonification terpene composition out of the reactor chamber and into downstream processing.

In certain embodiments, the sonification terpene composition is collected and pressure filtered (e.g., 220 microns to 25 microns) to isolate the cannabinoids. The oil/water mixtures may then be separated via any suitable methodology, e.g., centrifugation, decantation, etc., to form an extracted extracted bio-oil terpene product. If desired, the bio-oil product may be homogenized for uniformity, and further distilled and or concentrated for purity and potency. The resulting bio-oil product and powder is free of fractionation and extraction chemicals that are present in typical bio-oil products generated using chemical extraction methodologies.

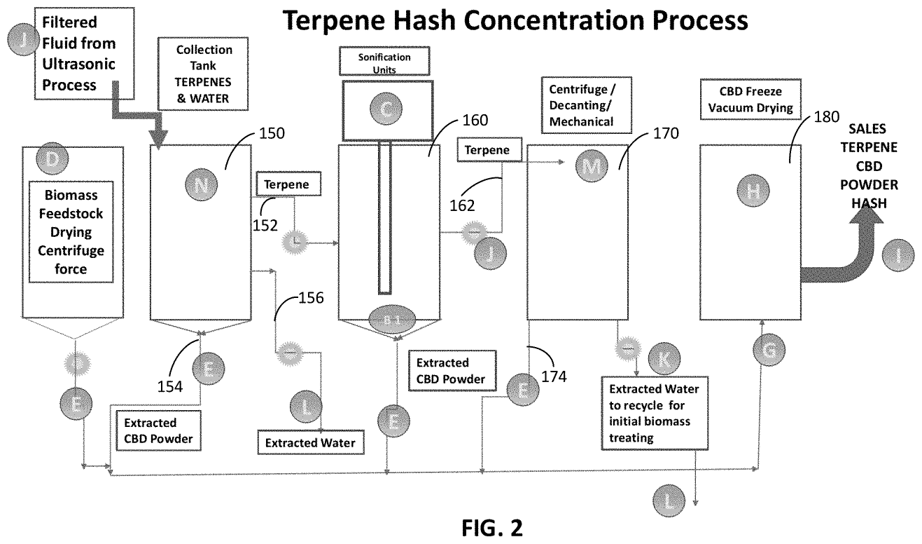

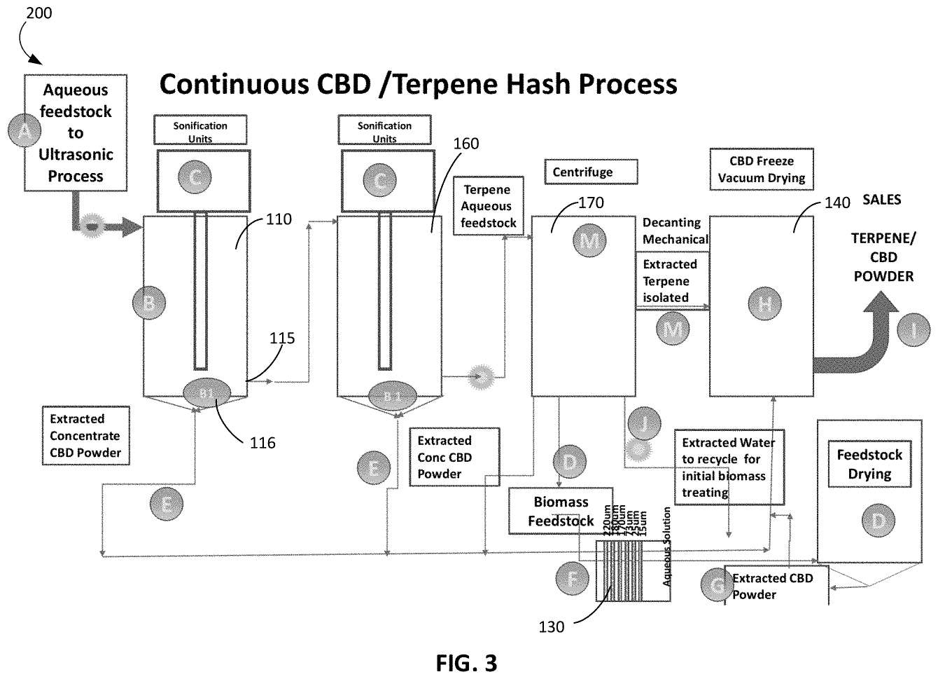

The methods of the disclosure may be performed in batch (FIG. 2) or continuous flow (FIG. 3) methodologies. In certain aspects, the methods of the disclosure may be used in connection with any suitable sonification apparatus known in the art. In certain embodiments, the sonification apparatus may comprise a sonification reactor that is scalable to increase capacity to accommodate desired biomass load.

In certain embodiments, with reference to FIG. 2, a batch method is exemplified, wherein the method comprise preparing a biomass feedstock at “A”, followed by providing the prepared biomass feedstock to a sonification reactor chamber at “B”. The biomass feedstock is then passed through the sonification reactor under sufficient conditions to result in biomass conversion to sonification volatiles, terpenes and biomass residue, i.e., a “sonification terpene composition”, at “C” (e.g., under temperature, slurry ratios, volume, and processing time). Remaining sonification biomass is removed from the sonification reactor chamber “B”, and transferred to a mechanical drying system at “D” to dehydrate. The sonification terpene composition (sonification volatiles, terpenes) are extracted from the bottom of the sonification reactor chamber at “E”, and filtered multiple times at “F”, e.g., utilizing 250 micron to 25 micron filter cloths, filter press or other suitable mechanical methods. The filtered solution is then mechanically and thermally separated to produce a filtered CBD composition (e.g., via centrifugal/disc separation; decanting; specific gravity; pour point changes; cooling; etc.) at “G”, and vacuum freeze dried at “H” to produce a bio-oil powder. The CBD composition may be further separated or concentrated as desired to result in a desired bio-oil powder product at “I”.

In certain aspects, the batch processing methodology of FIG. 2 may continue. By way of example, a terpene/aqueous mixture, may be recovered from the separation at “G”, and the terpene/aqueous mixture may be filtered at “J”. The filtered terpene/aqueous mixture may be transferred to a holding tank and gravity separated at “N”. Product is extracted from various levels (cannabinoids, water, terpene) for additional processing or recycling back to various process steps. For example, separated terpene solution may be recycled to “C” for sonification and further processing as described herein, e.g., mechanical separation to produce an extracted CBD composition (e.g., via centrifugal/disc separation; decanting; etc.) at “M”, with extraction of water at “K”. The water may be recycled to a refrigeration unit and then into subsequent batches of unprocessed biomass aqueous solution at “L”. As discussed above, the recovered terpene/cannabinoid compositions at “G” may be vacuumed freeze dried at “H” to produce bio-oil powders. The CBD composition may be further separated or concentrated as desired to result in a desired bio-oil powder product at “I”.

With reference to FIG. 2, provided is a non-limiting exemplary system 100 for extracting bio-oil and bio-powder from a biomass feedstock in accordance with aspects of the disclosure. System 100 may use prepared or non-prepared biomass feedstock. In certain embodiments, the biomass feed stock may be prepared by mechanical processing of biomass feed stock such as grinding, cutting, smashing, etc. Preferably, the biomass feed stock is solely prepared by mechanical processing. In other words, in at least one embodiment, the biomass feedstock is not processed, prepared, and/or altered using chemicals.

The biomass feedstock may be whole plant material or may be plant material obtained from select parts of the plant. For example, the plant material may be obtained from one or more of the flower, the leaf, the bud, the stem, and/or the root of the plant. In some instances, the biomass feedstock is raw agriculture plant, hemp, resin hemp, industrial hemp, hops or any other agricultural product. Non-limiting examples of biomass feedstock include at least one of resin hemp, industrial hemp, and cannabis. Preferably, the biomass feedstock comprises one or more cannabinoids including, but not limited to, THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), or CBT (cannabicitran). In some instances, biomass feedback contains a broad-spectrum of cannabinoids. Broad-spectrum cannabinoids, as used herein, refers to compositions comprising CBDA, CBN, CBG, CBC, CBL, CBV, THCV, CBDV, CBCV, CBGV, CBGM, CBE, and CBT.

Additionally or alternatively, the biomass feedstock includes at least one terpene compound. Without being limited by theory, it is believed that certain terpene compounds may provide therapeutic value and help create the “entourage effect.” The entourage effect refers to a beneficial effect obtained by numerous CBD compounds working together as opposed to just one or two of these compounds working in isolation. In certain instances, the biomass feedstock may include 50 or more, 60 or more, 70 or more, 80 or more, or 90 or more cannabinoid compounds. The biomass feedstock may include one or more flavonoids, such as flavonoids contained in the cannabis plant. There are about 20 flavonoids in cannabis, including catechins and quercetin. Without being limited to any specific theory, it is believed that flavonoids may provide beneficial effects including, e.g., anti-inflammatory, antioxidant, anti-fungal, anti-viral anti-bacterial, anti-cancer, and/or anti-allergic activity.

In some instances, system 100 is configured to produce a cannabinoid composition including combinations of CBD, beneficial terpenes, fatty acids, enzymes, and other phytocannabinoids naturally found in cannabis plants. In other instances, the cannabinoid composition consists of or consists essentially of CBD.

In certain embodiments, prior to preparation of the biomass feedstock, the biomass feedstock may be analyzed for various parameters, e.g., essential oil content, moisture content, chemical analysis, etc. As will be understood by those of skill in the art, such analysis of feedstock may be used to select and adjust sonification operating parameters, e.g., operating temperature, operating concentration (negative biomass/water ratio), processing time, etc. Such sonification operating parameters may be selected to provide sufficient conditions to result in biomass conversion to sonification volatiles and sonification biomass as the feedstock passes through the sonification reactor chamber.

In some embodiments, the biomass feedstock may comprise cannabis, and may be analyzed for cannabinoid content and moisture content. Example cannabinoids include, but are not limited to, THC, THCA, CBD, CBDA, CBN, CBG, CBC, CBL, CBV, THCV, CBDV, CBCV, CBGV, CBGM, CBE, CBT. In such embodiments, system 100 may be used to extract bio-oils comprising one or more cannabinoids, e.g., CBD oils.

System 100 includes a sonification unit 110 configured for the sonification of the biomass feedstock. The sonification apparatus may include a feedstock hopper and grinder/auger mixer upstream of the sonification unit 110, if desired, to facilitate loading of the biomass feedstock into sonification unit 110 via a biomass feedstock input port. In certain embodiments, the prepared biomass feedstock may be fed into a suitable loading mechanism, e.g., a rotating valve, which then feeds the feedstock into the sonification camber of sonification unit 110. The biomass water mixture is agitated using mechanical of fluid to ensure suspension and biomass contact time with the ultrasonication probe.

The sonification unit 110 may be operated under sufficient conditions (e.g., under temperature, slurry ratios, volume, and processing time) to result in biomass conversion to sonification volatiles, terpenes, and biomass residue. By way of non-limiting example, temperatures may range from about 20 to about 100° F., dependent on biomass feedstock analysis, frequencies may range from about 20 to about 30 KHz, power rating percentage and processing ratios may be between 25% to 75% biomass/water. The operating temperature generally varies in a reaction heating zone in the reactor chamber dependent on the sonic input and length of treatment. In some instances, the temperature may be from about 20 to about 100° F., about 20 to about 90° F., about 20 to about 80° F., about 20 to about 70° F., about 20 to about 60° F., about 20 to about 50° F., about 20 to about 40° F.; about 30 to about 100° F., about 30 to about 90° F., about 30 to about 80° F., about 30 to about 70° F., about 30 to about 60° F., about 30 to about 50° F.; about 40 to about 100° F. about 40 to about 90° F., about 40 to about 80° F., about 40 to about 70° F., about 40 to about 60° F.; about 50 to about 100° F. about 50 to about 90° F., about 50 to about 80° F., about 50 to about 70° F.; about 60 to about 100° F. about 60 to about 90° F., about 60 to about 80° F.; about 70 to about 100° F. or about 70 to about 90° F. Operating frequencies may generally be from the composition of the biomass feedstock analysis. For instances, the frequencies employed by sonification unit 110 may be from about 20 to about 30 KHz, about 20 to about 28 KHz, about 20 to about 26 KHz, about 20 to about 24 KHz; about 22 to about 30 KHz, about 22 to about 28 KHz, about 22 to about 26 KHz; about 24 to about 30 KHz, about 24 to about 28 KHz, or about 26 to about 30 KHz.

As illustrated in FIGS. 2 and 3, in some instances, sonification unit 110 includes a reaction chamber 111, an agitation mixer 112, a biomass feedstock input port 113, a solid fraction exit port 114, cannabinoid oil export 115, and cannabinoid solid export 116. In certain embodiments, sonification unit 110 may comprise a sonification reactor that is scalable to increase capacity to accommodate desired biomass load.

Sonification unit 110 may be configured so that as the biomass feedstock passes through the sonification reactor chamber, the biomass feedstock is sonically treated to form a cannabinoid volatile product and a biomass product. The sonification volatile product includes solid phase and a liquid phase. Gravity and centrifugal force within the sonification reactor chamber facilitate movement of the sonification volatile product out of the reactor chamber and into downstream processing. Sonification unit 110 is fluidically coupled to drying unit 120 and is fluidically coupled to filter unit 130.

Drying unit 120 is fluidically coupled to sonification unit 110 to receive stream 117 comprising sonification biomass. Drying unit 120 is configured for drying biomass, e.g., biomass received from the sonification unit 110. Generally, drying unit 120 may be selected from any suitable mechanical drying apparatuses that are capable of dehydrating biomass.

Filter unit 130 is fluidically coupled to sonification unit 110 to receive stream 118 comprising sonification volatiles (e.g., sonification volatiles, terpenes) from sonification unit 110. In some instances, filter unit 130 is fluidically coupled to the bottom of sonification unit 110. Filter unit 130 is typically configured to filter the sonification volatiles multiple times to separate the cannabinoids from a terpene/aqueous mixture by producing stream 132 comprising cannabinoids and stream 134 comprising a terpene/aqueous mixture. Filter unit 130 may, in certain embodiments, separate the oil/water mixtures suitable methodology, e.g., centrifugation, decantation, etc., to form an extracted bio-oil terpene product. Filter unit 130 may utilize filter cloths, filter press, or mechanical methods. For example, filter unit 130 may comprise a centrifugal filtering apparatus, a disc separation apparatus, a decanting apparatus, a plate and filter apparatus, or the like. Filter unit 130 may, additionally or alternatively, utilize apparatuses that are configured to filter by using gravity, thermal properties (pour point changes), and/or pressures. In at least one embodiment, filter unit 130 is configured to have filters rated for removing particles having a size of 25 microns to 250 microns. In another embodiment, a plurality of filters (e.g., a bubble bag) may be employed having one or more of the following filter ratings to remove particles having a size of greater than 220 microns, 180 microns, 120 microns, 73 microns, and/or 25 microns.

Freeze drying unit 140 is fluidically coupled to filter system 130, such that freeze drying unit 140 receives stream 132 comprising cannabinoids from filter unit 130. Freeze drying unit 140 may be configured for freeze drying the cannabinoids filtered out by filter unit 130. The cannabinoids may be further separated as desired to result in a desired bio-oil powder product.

Vessel 150 is typically fluidically coupled to filter unit 130 to receive stream 134 comprising terpene/aqueous composition from filter unit 130. Vessel 150 may be configured to gravity separate the terpene/aqueous composition. For instance, vessel 150 may be configured such that product (cannabinoids, water, terpene) is extracted from various levels of vessel 150 for further processing or recycling. As illustrated in the non-limiting embodiment of FIG. 1, vessel 150 produces stream 152 comprising terpenes, stream 154 comprising cannabinoids (e.g., CBD), and stream 156 comprising water. Vessel 150 can be fluidically coupled to sonification unit 160 to transfer stream 152 comprising terpenes to sonification unit 160.

Sonification unit 160 may be configured to separate cannabinoids from the terpene composition of the stream 152 received from vessel 150, and produce stream 162 comprising terpenes and stream 164 comprising cannabinoids. Sonification unit 160 may be similar to or different from sonification unit 120. In the embodiment illustrated in FIG. 1, sonification unit 160 is a centrifuge or decanting unit. Sonification unit 160 may be fluidically coupled to filter unit 170, such that filter unit 170 receives stream 162 comprising terpenes. Typically, stream 162 has a concentration of terpenes that is greater than the concentration of terpenes in stream 152.

Filter unit 170 may be similar to or different from filter unit 120. For example, filter unit 170 may comprise any of filtering apparatuses of filter unit 120, such as centrifugal filtering apparatuses, disc separation apparatuses, decanting apparatuses, plate and filter apparatuses, and/or the like. Filter unit 170 is typically configured to filter out terpenes, such that filter unit 170 produces stream 172 comprising terpenes and water and stream 174 comprising cannabinoids. The water may be further isolated and recycled, e.g., for treating or preparing the biomass feedstock.

System 100 may include freeze drying unit 180, which is configured to dry and, particularly, freeze dry, the compositions comprising cannabinoids. Freeze drying unit 180 is fluidically coupled to vessel 150, sonification unit 160, and filter unit 170 to receive streams 154, 164, and 174, which all comprise cannabinoids. The cannabinoids may be further separated as desired to result in a desired bio-oil powder product. In some embodiments, the cannabinoids are isolated and/or packaged after being freeze dried.

With reference to FIG. 3, a continuous extraction methodology in accordance with embodiments of the disclosure is shown. In accordance with the illustrated embodiments, processing steps similar to the batch processing of FIGS. 1 and 2 are illustrated, but the process will be mechanical, and material pumped and transported through the sonification chambers (two or more in series), mechanically separated in a three-phase centrifuge or disc separator machine, solution processed in a three phase centrifugal/disc separation/mechanical/decanting separated (specific gravity), thermally (pour point changes) filtered, cannabinoids “G” vacuum freeze dried “H”, and further separated as desired to result in a desired bio-oil powder product “I” to sales.

With reference to FIG. 2, provided is another non-limiting exemplary system 200 for extracting bio-oil from a biomass feedstock in accordance with aspects of the disclosure. System 200 is similar to system 100, but is configured for continuous extraction processes. The operation units of system 200 may be the same or similar to the operation units of system 100 and, thus, FIG. 2 includes similar reference numbers as FIG. 1. System 200 may include two or more sonification units 110 and 160 (e.g., operating in series), a first filter unit 170 (e.g., a three-phase centrifuge or disc separator machine), a second filter unit 130 (e.g. a frame and plate filter), and at least one freeze dryer unit 140.

In accordance with another aspect of the disclosure, provided is a cannabinoid product. The cannabinoid product may be produced using the methods and/or systems disclosed herein. Preferably, the cannabinoid product is free of harsh chemicals typically used during extraction process. In at least one embodiment, the cannabinoid product is free of or essentially free of chemicals, such as solvents. The cannabinoid product may be in an oil, tablet, capsule, or powder form. In certain embodiments, the cannabinoid product may be a bio-oil product and/or powder that is free of fractionation and extraction chemicals that are present in typical bio-oil products generated using chemical extraction methodologies.

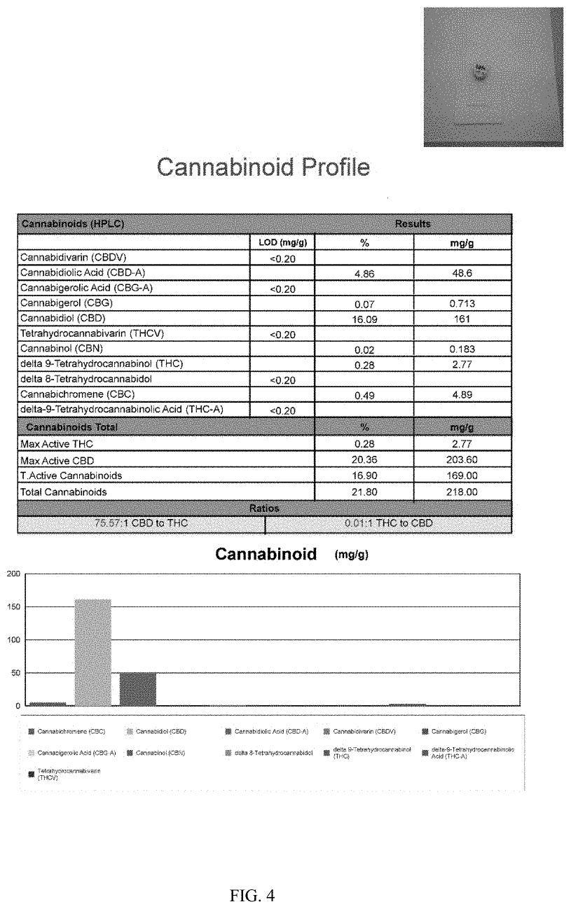

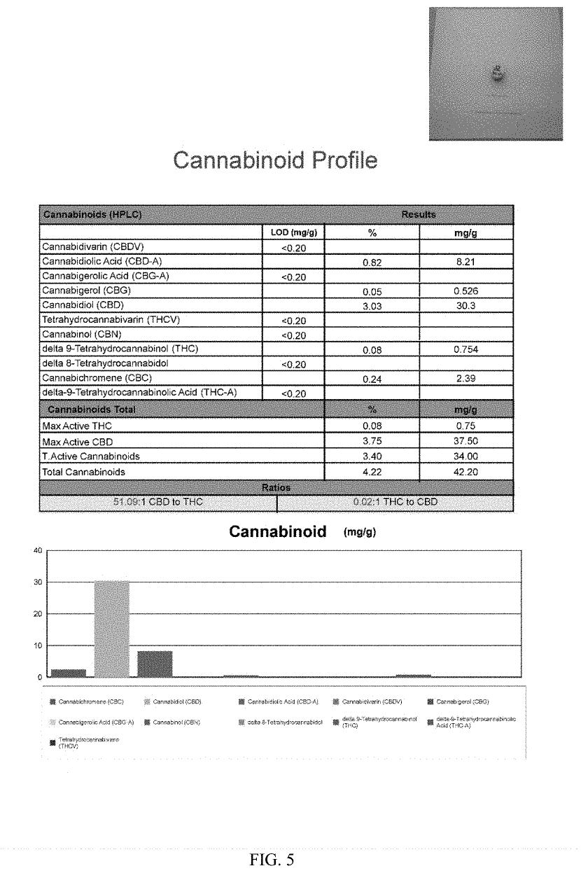

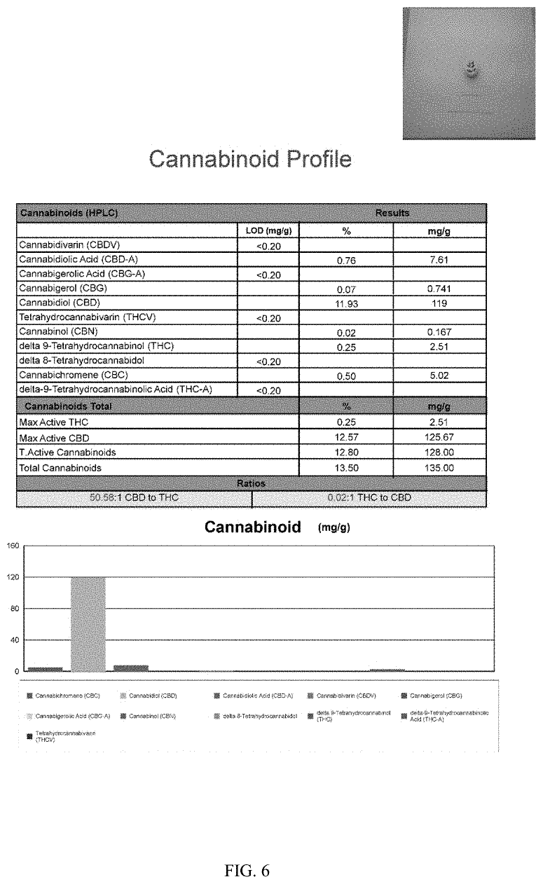

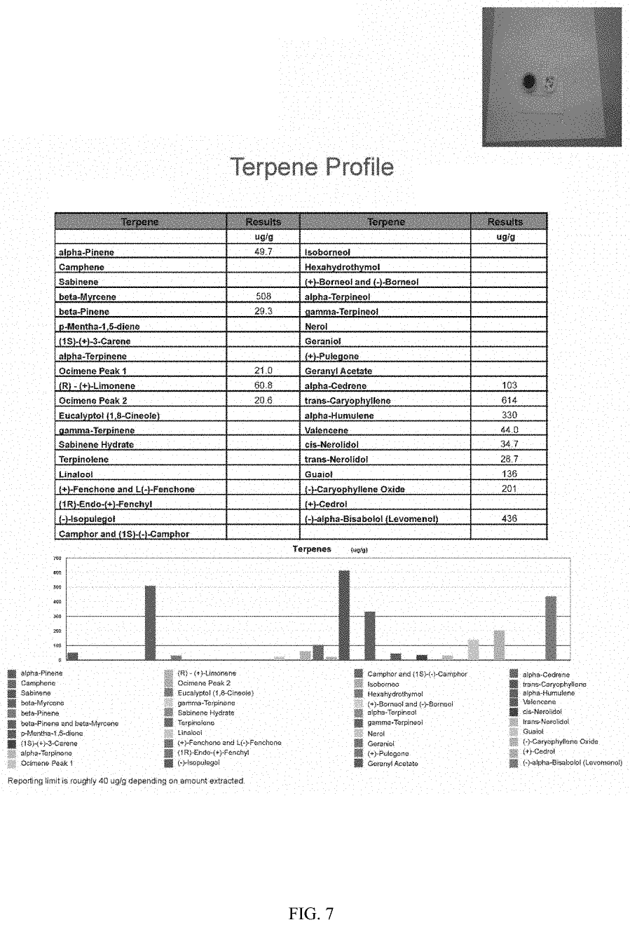

The cannabinoid product may comprise CBDA, CBN, CBG, CBC, CBL, CBV, THCV, CBDV, CBCV, CBGV, CBGM, CBE, CBT, terpenes, and/or flavonoids. In certain embodiments, the cannabinoid product comprises or consists of CBD, terpenes, and/or flavonoids. In some instances instances, the cannabinoid composition includes 50 or more, 60 or more, 70 or more, 80 or more, or 90 or more cannabinoid compounds or, preferably, CBD compounds. As seen in FIGS. 4-7, the cannabinoid product may have a cannabinoid profile comprising a plurality of cannabinoid compounds and/or a terpene profile comprising a plurality of terpenes. The cannabinoid profiles illustrated in FIGS. 4-6, desirably had a low amount of THC (e.g., 0.28%, 0.08%, and 0.25%, respectively), while having desirable amounts non-THC cannabinoids (e.g., 21.52%, 4.14%, and 13.25%, respectively). In the terpene profile of FIG. 7, the cannabinoid product included alpha-pinene, beta-myrcene, beta-pinene, ocimene peak 1, limonene, ocimene peak 2, alpha-cedrene, trans-caryophyllene, alpha-humulene, valencene, cis-nerolidol, trans-nerolidol, trans-Nerolidol, Guaiol, Caryophyliene Oxide, Cedrol, and alpha-bisabolol.

The amount of CBD, terpenes, and/or flavonoids in the cannabinoid product may vary, but are individually present in an amount typically from about 1 to about 99 wt. %, based on the total weight of the cannabinoid product. For example, the amount of CBD, terpenes, and/or flavonoids individually present in the cannabinoid product may be from about 1 to about 99 wt. %, about 1 to about 90 wt. %, about 1 to about 80 wt. %, about 1 to about 70 wt. %, about 1 to about 60 wt. % about 1 to about 50 wt. %, about 1 to about 40 wt. %, about 1 to about 30 wt. %, about 1 to about 20 wt. %, about 1 to about 15 wt. %, about 1 to about 10 wt. %; about 5 to about 99 wt. %, about 5 to about 90 wt. %, about 5 to about 80 wt. %, about 5 to about 70 wt. %, about 5 to about 60 wt. % about 5 to about 50 wt. %, about 5 to about 40 wt. %, about 5 to about 30 wt. %, about 5 to about 20 wt. %, about 5 to about 15 wt. %, about 5 to about 10 wt. %; about 10 to about 99 wt. %, about 10 to about 90 wt. %, about 10 to about 80 wt. %, about 10 to about 70 wt. %, about 10 to about 60 wt. % about 10 to about 50 wt. %, about 10 to about 40 wt. %, about 10 to about 30 wt. %, about 10 to about 20 wt. %, about 10 to about 15 wt. %; about 20 to about 99 wt. %, about 20 to about 90 wt. %, about 20 to about 80 wt. %, about 20 to about 70 wt. %, about 20 to about 60 wt. % about 20 to about 50 wt. %, about 20 to about 40 wt. %, about 20 to about 30 wt. %; about 30 to about 99 wt. %, about 30 to about 90 wt. %, about 30 to about 80 wt. %, about 30 to about 70 wt. %, about 30 to about 60 wt. % about 30 to about 50 wt. %, about 30 to about 40 wt. %; about 40 to about 99 wt. %, about 40 to about 90 wt. %, about 40 to about 80 wt. %, about 40 to about 70 wt. %, about 40 to about 60 wt. %, about 40 to about 50 wt. %; about 50 to about 99 wt. %, about 50 to about 90 wt. %, about 50 to about 80 wt. %, about 50 to about 70 wt. %, about 50 to about 60 wt. %; about 60 to about 99 wt. %, about 60 to about 90 wt. %, about 60 to about 80 wt. %, about 60 to about 70 wt. %; about 70 to about 99 wt. %, about 70 to about 90 wt. %, about 70 to about 80 wt. %; about 80 to about 99 wt. %, about 80 to about 90 wt. %; about 90 to about 99 wt. %, based on the total weight of the cannabinoid product.

Additionally or alternatively, the total amount of the combination of CBD, terpenes, and/or flavonoids in the cannabinoid composition may be from 1 to about 99 wt. %, based on the total weight of the cannabinoid product. In certain embodiments, the cannabinoid composition may have a total amount of CBD, terpenes, and/or flavonoids of about 10 to about 99 wt. %, about 10 to about 90 wt. %, about 10 to about 80 wt. %, about 10 to about 70 wt. %, about 10 to about 60 wt. % about 10 to about 50 wt. %, about 10 to about 40 wt. %, about 10 to about 30 wt. %, about 10 to about 20 wt. %, about 10 to about 15 wt. %; about 20 to about 99 wt. %, about 20 to about 90 wt. %, about 20 to about 80 wt. %, about 20 to about 70 wt. %, about 20 to about 60 wt. % about 20 to about 50 wt. %, about 20 to about 40 wt. %, about 20 to about 30 wt. %; about 30 to about 99 wt. %, about 30 to about 90 wt. %, about 30 to about 80 wt. %, about 30 to about 70 wt. %, about 30 to about 60 wt. % about 30 to about 50 wt. %, about 30 to about 40 wt. %; about 40 to about 99 wt. %, about 40 to about 90 wt. %, about 40 to about 80 wt. %, about 40 to about 70 wt. %, about 40 to about 60 wt. %, about 40 to about 50 wt. %; about 50 to about 99 wt. %, about 50 to about 90 wt. %, about 50 to about 80 wt. %, about 50 to about 70 wt. %, about 50 to about 60 wt. %; about 60 to about 99 wt. %, about 60 to about 90 wt. %, about 60 to about 80 wt. %, about 60 to about 70 wt. %; about 70 to about 99 wt. %, about 70 to about 90 wt. %, about 70 to about 80 wt. %; about 80 to about 99 wt. %, about 80 to about 90 wt. %; about 90 to about 99 wt. %, based on the total weight of the cannabinoid product. In at least one embodiment, the cannabinoid product includes a high concentration of CBD (e.g., at least 95%) in an isolated form, such as CBD isolate.

In certain instances, the cannabinoid product includes about 0.5 wt. % or less of THC, based on the total weight of the cannabinoid product. Preferably, the amount of THC present in the cannabinoid product is about 0.45 wt. % or less, about 0.4 wt. % or less, about 0.35 wt. % or less, about 0.3 wt. % or less, about 0.25 wt. % or less, about 0.2 wt. % or less, about 0.15 wt. % or less, or about 0.1 wt. % or less, based on the total weight of the cannabinoid product.

In certain embodiments, the methods of the disclosure produce bio-oil powders that comprise broad spectrum cannabinoids, e.g., including CBG, CBN, CBC, and CBD-A cannabinoids, as well as broad spectrum terpenes. In addition, CBD concentration of 15% to 25% from initial processing may be achieved (i.e., without additional concentration or distilling of oils. The methods do not require chemical additives, solvents or extraction agents, and therefore the resulting compositions are free from such harsh chemicals.

The methods and systems disclosed herein may utilize agriculture biomass to extract oils and powders from the plant to be used for other applications. The biomass feedstock may include any plant material that contains essential oils that are incorporated into lotions, balms, food, beverage or salves. Additionally the methods and systems herein can process larger feedstock volumes without additional labor being required. The methods and systems can efficiently extract Broad Spectrum cannabinoids from the large acreage feedstock grows. Additionally, the methods and systems disclosed herein are preferably scalable.

For powder production, the cannabis industry typically uses only the buds of the plant and the tricones are removed by freezing the tricones with ice water and the stirring of the water causes the tricones to fall off. The methods and systems disclosed herein for extracting CBD powder may, in some instances, use no chemicals or additives, with the final product analysis defined as a Broad Spectrum Cannabinoid powder. Full plant extracts contain, in addition to CBD, beneficial terpenes fatty acids, enzymes, and other phytocannabinoids naturally found in hemp plants.

Table 1 provides additional information relating to aspects of the disclosure. The section headings are used herein for organizational purposes only, and are not to be construed in any way as limiting the subject matter described.

TABLE 1

| ITEM | DEFINITION |
|---|---|
| Broad Spectrum | Broad-spectrum CBD contains all the cannabidiol and |
| Full Spectrum | terpenes the plant contains. These cannabidiols could include, |
| | CBDA (cannabidiolic acid), CBN (cannabinol), CBG |
| | (cannabigerol), CBC (cannabichromene), CBL (cannabicyclol), |
| | CBV (cannabivarin), THCV (tetrahydrocannabivarin), CBDV |
| | (cannabidivarin), CBCV (cannabichromevarin), CBGV |
| | (cannabigerovarin), CBGM (cannabigerol monomethyl ether), |
| | CBE (cannabielsoin), CBT (cannabicitran). CBD. Broad |
| | spectrum CBD contains all the cannabidiol and terpenes which |
| | have therapeutic value and help create the “entourage effect”. |
| | Full-spectrum CBD oil is the same as Broad spectrum but with |
| | THC included. Full spectrum oil contains ail the therapeutic |
| | value of Broad spectrum. |
| Cannabinoid profile | The concentration of active cannabinoids in a product or |
| | medication. |
| CBD Medical uses | CBD can provide relief for chronic pain, anxiety, |
| | inflammation, depression and multiple other conditions. |
| | CBD's may be potentially useful for: |
| | Autoimmune diseases (inflammation, rheumatoid |
| | arthritis) |
| | Neurological conditions (Alzheimer's, dementia, |
| | Parkinson's, multiple sclerosis, epilepsy, Huntington's |
| | chorea, stroke, traumatic brain injury) |
| | Metabolic syndrome (diabetes, obesity) |
| | Neuropsychiatric illness (autism, ADHD, PTSD, |
| | alcoholism) |
| | Gut disorders (colitis, Crohn's) |
| | Cardiovascular dysfunction (atherosclerosis, |
| | arrhythmia) |
| | Skin disease (acne, dermatitis, psoriasis) |
| CBD reaction in the body | Cannabidiol has effects on the brain. Without being |
| | limited to any specific theory, it is believed that |
| | cannabidiol may prevent the breakdown of a chemical in |
| | the brain that affects pain, mood, and mental function. |
| | Preventing the breakdown of this chemical and increasing |
| | its levels in the blood may potentially reduce psychotic |
| | symptoms associated with conditions such as |
| | schizophrenia. Cannabidiol might also block some the |
| | psychoactive effects of delta-9-tetrahydrocannabinol |
| | (THC). Also, cannabidiol is believed to to reduce pain and |
| | anxiety. |
| Disk separator- | The product to be clarified runs through a stationary inlet |
| Multi-phase | pipe into the interior of the bowl and is gently |
| | accelerated to full speed by the distributor. The |
| | disc stack in the bowl divides the product stream into |
| | many thin layers, thus creating a large surface area. |
| | Within the disc stack, the liquid mixture is separated, |
| | and the solids are discharged. |
| | The liquid phases separated by centrifugal force are |
| | discharged from the bowl under pressure via two |
| | centripetal pumps. Due to the strong centrifugal force, |
| | the separated solids settle at the edge of the bowl. The |
| | separated solids are expelled periodically at full speed by |
| | a hydraulic system located in the lower part of the bowl. |
| Entourage Effect | The beneficial effect of numerous CBD compounds |
| | working together as opposed to just one or two of these |
| | compounds working in isolation, all the terpenes, |
| | cannabinoids, flavonoids, and fatty acids found in hemp. |
| | Entourage Effect is the benefit you get from ingesting |
| | multiple components of the hemp. |
| Hemp | An industrial plant cultivated for its fiber and edible |
| Resin Hemp | seeds. While hemp is in the same family as the cannabis |
| Industrial Hemp | plant, it does not have psychoactive effects. Commercial |
| | items made from hemp fiber include paper, textiles, |
| | clothing, biodegradable plastic, and food. Hemp means |
| | the plant Cannabis sativa L. and any part of that plant, |
| | including the seeds thereof and all derivatives, extracts, |
| | cannabinoids, isomers, acids, salts, and salts of isomers, |
| | whether growing or not, with a delta-9 |
| | tetrahydrocannabinol concentration of not more than 0.3 |
| | percent on a dry weight basis, or as otherwise defined in |
| | federal law, whichever is more permissive. |
| | Hemp crop is one of more unprocessed Hemp plants or |
| | plant parts. |
| Kief | Kief refers to the resin glands which contain the terpenes |
| | and cannabinoids that make cannabis so unique. Kief |
| | refers to loosen trichromes, whereas hash has |
| | compressed trichromes. |
| Plate and Frame Filter | The plate and frame design, generally, utilize a flat, solid |
| Press or Recessed | plate (known as a “flush plate”) covered by a filter press |
| Chamber Filter Press | cloth, with a hollow frame between each plate. A metal |
| | skeleton holds and clamps the filter plates together to |
| | create a chamber, which is formed by the flush plate on |
| | each side of a frame. Filter cake is collected within the |
| | frame. |
| | The slurry is pumped into a comer hole and flows into |
| | each frame, allowing solid particles to accumulate on the |
| | filter cloths. The remaining filtered liquid (also known as |
| | filtrate) then moves to a drainage port in the flush plate |
| | and into a comer hole that is not being used for feeding |
| | the slurry. The filtrate then travels to discharge piping |
| | and is directed to the next step in the process. |
| | After a volume of mixture is pumped, the frames become |
| | filled with solids, the slurry feed pump turns off, and the |
| | filter press is ready to open. Each frame should now |
| | contain a filter cake, which is the result of the solids |
| | forming on the filter cloths. The filter cakes are then |
| | scraped out of the frames using a spatula, ideally falling |
| | into a cake hopper placed below the press.. |
| | Generally, in a process where immediate particle |
| | retention is important, filter paper may be used as fine |
| | solids would either blind off the filter cloth or simply |
| | pass through it. Note that filter cloths may still be used to |
| | support the filter paper. The filter cloths may be |
| | reusable, but the filter paper is typically replaced after |
| | every filtration cycle. |
| | Polishing applications are sized for a specific filtration |
| | area based on the process flow rate and the known flux |
| | rate (volume per area per unit of time). To avoid dips in |
| | achievable filtration flow as the press fills with solids, |
| | polishing applications msy include a redundant filter |
| | press that is also sized to accept 100% of the desired |
| | process flow. |
| | A recessed chamber filter press utilizes filter plates that |
| | form a series of cavities between them when they are |
| | clamped together. Suspended particulates collect on the |
| | filter cloths and build up inside each cavity space to form |
| | a filter cake. Compare this to the hollow frames of the |
| | plate and frame design where solids are collected. |
| Pour Point | Pour point is the temperature above which a lubricant or |
| | fluid will move freely under normal conditions. The |
| | pour point of a liquid is the temperature below which the |
| | liquid loses its flow characteristics. It is defined as the |
| | minimum temperature in which the oil can pour down |
| | from a beaker. |
| Sonification | Sonication units may be configured to apply sound |
| Ultra-sonification | energy to agitate particles in a sample, for various |
| | purposes, such as the extraction of multiple compounds |
| | from the biomass feedback. Ultrasonic frequencies |
| | (>20 kHz) are may be used. Sonication may have |
| | multiple effects, e.g., chemical effects, physical effects, |
| | or a combination thereof. |
| Specific gravity | Specific gravity, also called relative density, is the ratio |
| | of the density of a substance to the density of a reference |
| | substance; equivalently, it is the ratio of the mass of a |
| | substance to the mass of a reference substance for the |
| | same given volume. For fluids, the reference substance is |
| | usually the water, with a density of 1.00 × 10³ kg/m³ at 4 |
| | degrees Celsius (water's densest temperature), used to |
| | determine whether the fluid will sink or float in water. |
| | Water is usually assumed to be the reference substance |
| | with liquids. |
| Terpenes | Terpenes are the aromatic compounds found in plants. |
| | Common terpenes are limonene (found in citrus fruits) |
| | and linalool (found in lavender). Terpenes may have |
| | therapeutic effects on the human body. |
| Tetrahydrocannabinol | The main active psychoactive constituent of cannabis. It |
| (THC) | is responsible for the high sensation. |
| Tincture | A liquid that contains a concentrated herbal extract. A |
| | mixture of CBD and a different herbal extract. |

All publications, patents and patent applications are herein incorporated by reference to the same extent as if each individual publication, patent or patent application was specifically and individually indicated to be incorporated by reference.

The terms “comprising,” “having,” “including” and “containing” are used in their open, non-limiting sense. The terms “a” and “the” are understood to encompass the plural as well as the singular. The compositions and methods of the present disclosure can comprise, consist of, or consist essentially of the essential elements and limitations of the disclosure described herein, as well as any additional or optional ingredients, components, or limitations described herein or otherwise useful. As used herein, the expression “at least one” is interchangeable with the expression “one or more” and thus includes individual components as well as mixtures/combinations.

All percentages, parts and ratios herein are based upon the total weight of the compositions of the present disclosure, unless otherwise indicated. All ranges and values disclosed herein are inclusive and combinable. The expression “inclusive” for a range of concentrations means that the limits of the range are included in the defined interval. For examples, any value or point described herein that falls within a range described herein can serve as a minimum or maximum value to derive a sub-range, etc. Furthermore, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Other than in the operating examples, or where otherwise indicated, all numbers expressing quantities of ingredients and/or reaction conditions are to be understood as being modified in all instances by the term “about,” meaning within +/−5% of the indicated number.

The term “substantially free” or “essentially free” as used herein means that there is less than about 5% by weight of a specific material or compound within the respective stream and/or cannabinoid composition, based on the total weight of the compositions. Nonetheless, the compositions may include less than about 2 wt. %, less than about 1 wt. %, less than about 0.5 wt. %, less than about 0.1 wt. %, less than 0.01 wt. %, or none of the specified material.

Throughout the disclosure, the terms “a mixture thereof” or “a combination thereof” may be used following a list of elements as shown in the following example where letters A-F represent the elements: “one or more elements selected from the group consisting of A, B, C, D, E, F, and a mixture thereof” The term, “a mixture thereof” or “a combination thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included. The salts referred to throughout the disclosure may include salts having a counter-ion such as an alkali metal, alkaline earth metal, or ammonium counter-ion. This list of counter-ions, however, is non-limiting.

Additionally, all components and elements positively set forth in this disclosure can be negatively excluded from the claims.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### Examples

Exemplary Method—tepene and cannabinoid profiles such as those of FIGS. 4-7 may be obtained using a methodology as illustrated.
Feed stock analyzed for CHB and moisture content
   Analysis determines—Temperature, processing time, frequency, power rating and biomass ratio that will be required
Feedstock chopped into small pieces chop, cut, grind to fit into machine
Stainless Steel vessels mixed approximately 3 parts aqueous solution to 1 part hemp
   Dependent on feedstock analysis
   Temperature of aqueous solution varied from analysis
   Length of ultrasonic treatment varied from feedstock analysis—Varied from
   10 min to 45 min.
   Frequency and power required of ultrasonic equipment
      20-30 Mh
      40% to 95% power
   Extracted liquid and cannabinoids to vacuum filter press
      Filter size from 250 micron to 25 microns
   Filter pressed cannabinoids to vacuum freeze dryer
Feedstock fed into centrifuge
   Dry feedstock and recover aqueous solution
Terpene/water mixture processed
   Separate oils from plant water multiple filters
      Filter sizes
         Bubble bag: rinse hash through filters below
         220 microns
         180 microns
         120 microns
         73 microns
         25 microns—air dry and put in freezer
         <25 microns—left over terpene liquid->
   Recovered fluid to centrifuge
      Remove clear fluid from filter
         Recycle aqueous solution into next feedstock
      Remove terpene fluid
         Dehydrate terpene fluid mechanically or with heat
         Recovered Solid terpene CBD to vacuum freeze dryer
   Filtered sediment to vacuum freezer dryer
Prepare chemical free product

What is claimed is:

1. A method for obtaining a cannabinoid powder comprising a filtered CBD composition, the method comprising:
(a) providing a biomass feedstock comprising a portion of Cannabis sativa plant;
(b) extracting by sonification of the biomass feedstock at least a sonification terpene composition; and
(c) filtering the sonification terpene composition using a pressure filter to produce a filtered CBD composition and a filtered terpene mixture, and
(d) forming the cannabinoid powder from at least the filtered CBD composition.

2. The method of claim 1 further comprising:
(d) drying the filtered CBD composition of (c) to form a CBD powder.

3. The method of claim 1 further comprising:
(e) separating the filtered terpene mixture of (c) to produce at least one of a second CBD composition, a second terpenes composition, water, or a combination thereof.

4. The method of claim 1, wherein the pressure filter comprises at least two filtration stages selected from the group consisting of: greater than 220 microns, greater than 180 microns, greater than 120 microns, greater than 73 microns, and greater than 25 microns.

5. The method of claim 1, wherein the biomass feed stock comprises at least one of Cannabis sativa flower, Cannabis sativa stems, Cannabis sativa bud, or combinations thereof.

6. The method of claim 1, wherein the biomass feedstock is extracted by sonification for at least 10 minutes to least 45 minutes.

7. The method of claim 1, wherein the pressure filter is a plate and frame filter.

8. The method of claim 2, wherein the filtered CBD is vacuum freeze-dried to form the CBD powder.

9. A cannabinoid powder produced by the method comprising:
(a) providing a biomass feedstock comprising a portion of Cannabis sativa plant;
(b) extracting by sonification of the biomass feedstock at least a sonification terpenes composition; and
(c) filtering the sonficiation terpenes composition using a pressure filter to produce a filtered CBD composition and a filtered terpene mixture, and
(d) forming the cannabinoid powder from at least the filtered CBD composition.
wherein the cannabinoid powder comprises at least 15 wt. % of CBD.

10. The cannabinoid powder of claim 8, wherein the biomass feedstock does not comprise any chemical solvents.
